(19) United States
(12) Patent Application Publication
(10) Pub. No.: US 2022/0356071 A1
(43) Pub. Date: Nov. 10, 2022

(54) PROCESS FOR PREPARING COATED ZINC OXIDE PARTICLES BY FLAME SPRAY PYROLYSIS

(71) Applicant: L'OREAL, Paris (FR)

(72) Inventors: Valérie JEANNE-ROSE, AULNAY-SOUS-BOIS (FR); Henri SAMAIN, CHEVILLY-LARUE (FR); Yiannis DELIGIANNAKIS, IOANNINA (GR); Maria LOULOUDI, IOANNINA (GR)

(73) Assignee: L'OREAL, Paris (FR)

(21) Appl. No.: 17/789,488

(22) PCT Filed: Dec. 24, 2020
(86) PCT No.: PCT/EP2020/087873

§ 371 Date: Jun. 27, 2022

(30) Foreign Application Priority Data
Dec. 27, 2019 (FR) 1915678

(51) Int. Cl. C01G 9/02; C09C 1/04; C09D 7/62; C01B 13/34

(52) U.S. Cl. CPC C01G 9/02; C09C 1/043; C09D 7/62; C01B 13/34; C01P 2004/62; C01P 2004/64

(57) ABSTRACT

The present invention relates to a process for preparing coated zinc oxide particles by means of flame spray pyrolysis technology, to coated zinc oxide particles, and to a composition comprising said particles. The present invention also relates to specific zinc oxide particles derived from such a process, to the compositions comprising such particles and also to the uses thereof.

The present invention relates to a process for preparing coated zinc oxide particles by means of flame spray pyrolysis technology, to coated zinc oxide particles, and to a composition comprising said particles.

The present invention also relates to specific zinc oxide particles derived from such a process, to the compositions comprising such particles and also to the uses thereof.

Zinc oxide is used in many applications (cosmetics, paints, stains, electronics, rubber, etc.), notably for its optical properties. In particular, use is made of its light absorption and/or light scattering properties in order to protect surfaces from UV radiation and/or in order to convert ambient light into electricity.

However, zinc oxide has the drawback of being particularly unstable over time. More particularly, zinc oxide may degrade to zinc hydroxide, or even to Zn²⁺ ion, in the presence of water originating from the composition comprising it or from atmospheric moisture. Such a degradation leads to a partial or even total solubilization of the zinc oxide in water and has the effect of greatly reducing, or even removing, the desired properties of the zinc oxide.

This instability is particularly problematic when zinc oxide is used in photoprotective cosmetic compositions. Indeed, the ultraviolet radiation protection decreases as the zinc oxide degrades.

It has been envisaged to coat the zinc oxide with silica using sol-gel processes or else to graft fluoro compounds onto the zinc oxide. However, these solutions are not entirely satisfactory. The zinc oxide coated with silica via a sol-gel process generally has worse optical properties than an uncoated particle. As for the grafting technique, the use of fluoro compounds may be harmful to the environment and dangerous for the user.

It is also known to use a flame spray pyrolysis method (FSP method) to prepare zinc oxide particles.

Flame spray pyrolysis or FSP is a well-known method these days, which was essentially developed for the synthesis of ultrafine powders of single or mixed oxides of various metals (e.g. SiO₂, Al₂O₃, B₂O₃, ZrO₂, GeO₂, WO₃, Nb₂O₅, SnO₂, MgO, ZnO), with controlled morphologies, and/or the deposition thereof on various substrates, by starting from a wide variety of metal precursors, generally in the form of organic or inorganic, preferably inflammable, sprayable liquids; the liquids sprayed into the flame, by being burnt, notably emit nanoparticles of metal oxides which are sprayed by the flame itself onto these various substrates. The principle of this method has been recalled for example in the recent (2011) publication by Johnson Matthey entitled “Flame Spray Pyrolysis: a Unique Facility for the Production of Nanopowders”, Platinum Metals Rev., 2011, 55, (2), 149-151. Numerous variants of FSP processes and reactors have also been described, by way of example, in the patents or patent applications: U.S. Pat. Nos. 5,958,361, 2,268,337, WO 01/36332 or U.S. Pat. No. 6,887,566, WO 2004/005184 or U.S. Pat. No. 7,211,236, WO 2004/056927, WO 2005/103900, WO 2007/028267 or U.S. Pat. No. 8,182,573, WO 2008/049954 or U.S. Pat. No. 8,231,369, WO 2008/019905, US 2009/0123357, US 2009/0126604, US 2010/0055340, WO 2011/020204.

However, this method, applied to the preparation of zinc oxide can still be perfected, notably in order to improve the stability of the zinc oxide particles over time, and more particularly, its water resistance.

There is therefore a real need to develop a process for preparing zinc oxide particles that makes it possible to give said particles a good stability over time, and very particularly a good water resistance, while preserving good optical properties in terms of absorption and/or scattering of light, more particularly of ultraviolet radiation.

These aims are achieved by the present invention, one subject of which is notably a process for preparing coated zinc oxide particles, in particular of Zn-M oxide type, characterized in that it comprises at least the following steps:
a. preparing a composition (A) by adding one or more zinc precursors to a combustible solvent or to a mixture of combustible solvents; then
b. in a flame spray pyrolysis device, forming a flame by injecting the composition (A) and an oxygen-containing gas until aggregates of zinc oxide are obtained; and
c. injecting into the flame a composition (B) comprising one or more precursors of element M and one or more solvents until an (in)organic, preferably inorganic, coating layer containing at least one element M and at least one oxygen atom is obtained on the surface of said zinc oxide aggregates; said element M being chosen from elements from column 4, elements from column 13 and elements from column 14 of the Periodic Table of the Elements.

It has been observed that the process according to the invention makes it possible to obtain zinc oxide particles coated with a layer of inorganic material based on the element M, which are particularly stable over time and have a good water resistance.

Furthermore, unlike conventional coating processes, the process according to the invention has the advantage, despite the presence of the coating, of retaining good intrinsic properties of the centre. Indeed, owing to the specific nature of the coating layer, it is possible, for a given particle weight, to reduce the proportion of zinc oxide, without however reducing and/or negatively affecting the properties of said zinc oxide.

Thus, the process of the invention makes it possible to produce stable zinc oxide particles, while avoiding the inconveniences owing to the increase in the amount of particles which would be conventionally necessary in order to maintain the good optical properties of the zinc oxide.

These zinc oxide particles, in particular of Zn-M oxide type, comprise a core 1 and one or more upper coating layers 2 covering said core 1, and are characterized in that:
(i) the core 1 consists of zinc oxide, preferably in the crystalline state;
(ii) the upper coating layer(s) 2 cover at least 90% of the surface of the core 1, preferably cover the whole of the surface of the core 1, and comprise one or more (in)organic, preferably inorganic, compounds containing one or more elements M and one or more oxygen atoms;
(iii) said element(s) M are chosen from elements from column 4, elements from column 13 and elements from column 14 of the Periodic Table of the Elements;
   and it being understood that:
   when said element(s) M are silicon then the (Zinc/Silicon)_{particle} molar atomic ratio is strictly less than 2, and
   when said element(s) M are different from silicon then the (Zinc/M)_{particle} molar atomic ratio is within the range extending from 0.1 to 10; and
   the BET specific surface area of the particle is between 1 m²/g and 350 m²/g.

More particularly, the coated zinc oxide particles according to the invention only deteriorate very little over time in the presence of water, even when they are formulated in an aqueous composition.

It has also been observed that the zinc oxide particles prepared according to the invention have good optical properties in terms of light absorption and/or light scattering. More particularly, they have a high UV absorption and a low visible scattering or a high visible scattering, then allowing uses such as sun protection and/or modification of the visual appearance, while benefiting from resistance in the presence of water.

Moreover, the compositions comprising coated zinc oxide particles according to the invention have shown a good screening power, notably with respect to long and short UV-A radiation.

Furthermore, the compositions comprising the coated zinc oxide particles of the invention have an especially high transparency, which may prove advantageous when the composition is applied then left to dry on the coating, and in particular on the skin.

Moreover, since the coated zinc oxide particles according to the invention do not require a hydrophobic coating, it is possible to use them over a broad formulation spectrum (for example, in entirely aqueous formulations and/or surfactant-free formulations). When the formulations thus obtained end up in water (washbasin drainage, lake or sea), the risk of inappropriate deposit (on the edges of the washbasin, on the walls of the pipes or on rocks) is furthermore reduced.

### BRIEF DESCRIPTION OF THE FIGURES

The attached drawings are schematic. The drawings are not necessarily to scale; they are above all intended to illustrate the principles of the invention. In these drawings, from FIG. 1 to FIG. 2, elements (or parts of an element) that are identical are identified by the same reference signs.

FIG. 1 represents a cross-sectional view of a zinc oxide particle according to one embodiment of the invention.

FIG. 2 represents a cross-sectional view of a zinc oxide particle also covered with an additional coating layer according to another embodiment of the invention.

Other subjects, features, aspects and advantages of the invention will emerge even more clearly on reading the description and the example that follows.

In the present description, and unless otherwise indicated:
the expression “at least one” is equivalent to the expression “one or more” and can be replaced therewith;
the expression “between” is equivalent to the expression “ranging from” and can be replaced therewith, and implies that the limits are included;
the expression “keratin materials” denotes in particular the skin and also human keratin fibres such as the hair;
the core (1) is also referred to as the “centre”;
the upper coating layers (2) are also referred to as “outer layers”, “shell” or “coating”;
the expression “(in)organic compounds” is equivalent to “organic or inorganic compounds”;
an “alkyl” is understood to mean an “alkyl radical”, i.e. a C₁ to C₁₀, particularly C₁ to C₈, more particularly C₁ to C₆, and preferentially C₁ to C₄, linear or branched hydrocarbon-based radical, such as methyl, ethyl, propyl, isopropyl, n-butyl, isobutyl or tert-butyl;
an “aryl” radical is understood to mean a monocyclic or polycyclic, fused or non-fused carbon-based group, comprising from 6 to 22 carbon atoms, at least one ring of which is aromatic; preferentially, the aryl radical is a phenyl, biphenyl, naphthyl, indenyl, anthracenyl or tetrahydronaphthyl, preferably a phenyl;
an “arylate” radical is understood to mean an aryl group which comprises one or more —C(O)O⁻ carboxylate groups, such as naphthalate or naphthenate;
“complexed zinc” is understood to mean that the zinc forms a “metal complex” or “coordination compounds” in which the metal ion, corresponding to the central atom, i.e. zinc, is chemically bonded to one or more electron donors (ligands);
a “ligand” is understood to mean a coordinating organic chemical group or compound, i.e. which comprises at least one carbon atom and which is capable of coordinating with a metal, notably the Zn atom, preferably Zn(II) and which, once coordinated or complexed, results in metal compounds corresponding to principles of a coordination sphere with a predetermined number of electrons (internal complexes or chelates)—see Ullmann's Encyclopedia of Industrial Chemistry, “Metal complex dyes”, 2005, p. 1-42. More particularly, the ligand(s) are organic groups which comprise at least one group that is electron-donating via an inductive and/or mesomeric effect, more particularly bearing at least one amino, phosphino, hydroxy or thiol electron-donating group, or the ligand is a persistent carbene, particularly of “Arduengo” type (imidazol-2-ylidenes) or comprises at least one carbonyl group. As ligand, mention may more particularly be made of: i) those which contain at least one phosphorus atom —P< i.e. phosphine such as triphenyl phosphines; ii) bidendate ligands of formula R—C(X)—CR′R″—C(X)—R′″ with R and R″″, which are identical or different, representing a linear or branched (C₁-C₆)alkyl group, and R′ and R″, which are identical or different, representing a hydrogen atom or a linear or branched (C₁-C₆)alkyl group, preferentially R′ and R″ represent a hydrogen atom, X represents an oxygen or sulfur atom, or an N(R) group with R representing a hydrogen atom or a linear or branched (C₁-C₆)alkyl group, such as acetylacetone or β-diketones; iii) (poly)hydroxy carboxylic acid ligands of formula [HO—C(O)]n-A-C(O)—OH and the deprotonated forms thereof with A representing a monovalent group when n has the value zero or a polyvalent group when n is greater than or equal to 1, which is saturated or unsaturated, cyclic or non-cyclic and aromatic or non-aromatic based on a hydrocarbon comprising from 1 to 20 carbon atoms which is optionally interrupted by one or more heteroatoms and/or is optionally substituted, notably with one or more hydroxyl groups: preferably, A represents a monovalent (C₁-C₆)alkyl group or a polyvalent (C₁-C₆)alkylene group optionally substituted with one or more hydroxyl groups; and n representing an integer between 0 and 10 inclusive; preferably, n is between 0 and 5, for instance among 0, 1 or 2; such as lactic, glycolic, tartaric, citric and maleic acids, and arylates such as naphthalates; and iv) C₂ to C₁₀ polyol ligands, comprising from 2 to 5 hydroxyl groups, notably ethylene glycol, glycerol, more particularly still the ligand(s) bear a carboxy, carboxylate or amino group, particularly the ligand is chosen from acetate, (C₁-C₆)alkoxylate, (di)(C₁-C₆)alkylamino, and arylate, such as naphthalate or naphthenate, groups;

The term “fuel” is understood to mean a liquid compound which, with dioxygen and energy, is burnt in a chemical reaction generating heat: combustion. In particular the liquid fuels are chosen from protic solvents, in particular alcohols such as methanol, ethanol, isopropanol, n-butanol; aprotic solvents in particular chosen from esters such as methyl esters and those derived from acetate, such as 2-ethylhexyl acetate, acids such as 2-ethylhexanoic acid (EHA), acyclic ethers such as ethyl ether, methyl tert-butyl ether (MTBE), methyl tert-amyl ether (TAME), methyl tert-hexyl ether (THEME), ethyl tert-butyl ether (ETBE), ether tert-amyl ether (TAEE), diisopropyl ether (DIPE), cyclic ethers such as tetrahydrofuran (THF), aromatic hydrocarbons or arenes such as xylene, non-aromatic hydrocarbons; and mixtures thereof.

The fuels may optionally be chosen from liquefied hydrocarbons such as acetylene, methane, propane or butane; and mixtures thereof.

The Process for Preparing the Coated Zinc Oxide Particles

The preparation process according to the invention comprises a step (a) of preparing a composition (A) containing one or more zinc precursors and one or more combustible solvents.

The zinc precursors and the combustible solvents that can be used according to the invention may be chosen from the zinc precursors and the combustible solvents conventionally used in flame spray pyrolysis.

Preferably, the zinc precursor included in the composition (A) comprises one or more zinc atoms optionally complexed with one or more ligands containing at least one carbon atom.

More preferentially, said ligand(s) are chosen from acetate, (C₁-C₆)alkoxylate, (di)(C₁-C₆)alkylamino, and arylate, such as naphthalate or naphthenate, groups.

Preferably, the combustible solvent(s) are chosen from protic combustible solvents, aprotic combustible solvents, and mixtures thereof; more preferentially from alcohols, esters, acids, acyclic ethers, cyclic ethers, aromatic hydrocarbons or arenes, non-aromatic hydrocarbons, and mixtures thereof; and better still from 2-ethylhexyl acetate, 2-ethylhexanoic acid (EHA), ethyl ether, methyl tert-butyl ether (MTBE), methyl tert-amyl ether (TAME), methyl tert-hexyl ether (THEME), ethyl tert-butyl ether (ETBE), ether tert-amyl ether (TAEE), diisopropyl ether (DIPE), tetrahydrofuran (THF), xylene, and mixtures thereof.

More preferentially, the combustible solvent(s) are chosen from aprotic combustible solvents comprising at least three carbon atoms and mixtures thereof; more preferentially still from xylene, tetrahydrofuran, 2-ethylhexyl acetate, 2-ethylhexanoic acid (EHA), and mixtures thereof.

Advantageously, the content of zinc precursor in composition (A) is between 1% and 60% by weight and preferably between 15% and 30% by weight relative to the total weight of composition (A).

The preparation process according to the invention further comprises a step (b) of injecting composition (A) and an oxygen-containing gas into a flame spray pyrolysis (FSP) device to form a flame.

During this step (b), composition (A) and the oxygen-containing gas are advantageously injected into the flame spray pyrolysis device, by two injections that are separate from one another. In other words, composition (A) and the oxygen-containing gas are injected separately, i.e. composition (A) and the oxygen-containing gas are not injected by means of a single nozzle.

More particularly, composition (A) is transported by one tube, whereas the oxygen-containing gas (also referred to as “dispersion Oxygen”) is transported by another tube. The inlets of the two tubes are arranged so that the oxygen-containing gas produces a negative pressure and, via a Venturi effect, causes the composition (A) to be sucked up and converted into droplets.

Step (b) may optionally further comprise an additional injection of a “premix” mixture comprising oxygen and one or more combustible gases. This “premix” mixture is also referred to as “supporting flame oxygen” and enables the production of a support flame intended to ignite and maintain the flame resulting from composition (A) and the oxygen-containing gas (i.e. “dispersion Oxygen”).

Preferably, during step (b), composition (A), the oxygen-containing gas and optionally the “premix” mixture when it is present, are injected into a reaction tube, also referred to as an “enclosing tube”. Preferably, this reaction tube is made of metal or of quartz. Advantageously, the reaction tube has a height of greater than or equal to 30 cm, more preferentially greater than or equal to 40 cm, and better still greater than or equal to 50 cm. Advantageously, the length of said reaction tube is between 30 cm and 300 cm, preferably between 40 cm and 200 cm, more preferentially between 45 cm and 100 cm, and better still this length is equal to 50 cm.

The weight ratio of the mass of solvent(s) present in composition (A) on the one hand, to the mass of oxygen-containing gas on the other hand, is defined as follows: Firstly, the amount of oxygen-containing gas (also referred to as oxidizer compound) is calculated in order for the assembly formed by composition (A), i.e. the combustible solvent(s) and the zinc precursor(s) on the one hand, and the oxygen-containing gas on the other hand, to be able to react together in a combustion reaction in a stoichiometric ratio (therefore without an excess or deficit of oxidizer compound).

Starting from this calculated amount of oxygen-containing gas, also referred to as “calculated oxidizer”, a new calculation is performed to deduce therefrom the amount of oxygen-containing gas to be injected, also referred to as “oxidizer to be injected”, according to the formula: Oxidizer to be injected=Calculated oxidizer/φ with φ preferably between 0.30 and 0.9, and more preferentially between 0.4 and 0.65.

This method is notably defined by Turns, S. R. in An Introduction to Combustion: Concepts and Applications, 3rd ed.; McGraw-Hill: New York, 2012.

The preparation process according to the invention further comprises a step (c) of injecting, into the flame formed during step (b), a composition (B) comprising one or more solvents and one or more precursors of element M; said element M being chosen from elements from column 4, elements from column 13 and elements from column 14 of the Periodic Table of the Elements.

In other words, the process of the invention is continuous and the flame formed in step (b) is maintained.

During step (c) of the preparation process of the invention, the compositions (A) and (B) are injected separately and simultaneously. In other words, composition (A) is transported by one tube, whereas composition (B) is transported by another tube. The distance between the outlet of the two tubes is preferably at least 30 cm, and more preferentially at least 40 cm.

Preferably, the flame formed during step (b) is at a temperature above or equal to 2000° C., in at least one part of the flame.

At the site of the injection of the composition (B) into the flame formed in step (b) and maintained in step (c), i.e. during step (c), the temperature is preferably between 200° C. and 600° C., and more preferentially between 300° C. and 400° C.

Advantageously, during step c), composition (B) is injected via a spraying ring, placed above said reaction tube as described above, where in particular the injection of composition (A) takes place.

Preferably, the element M precursor comprises at least two M atoms and several M-carbon covalent bonds. More preferentially, the element M precursor comprises at least three M atoms and several M-carbon covalent bonds.

The element(s) M that can be used according to the invention are chosen from elements from column 4 (titanium column), elements from column 13 (boron column) and elements from column 14 (carbon column) of the Periodic Table of the Elements.

In other words, the elements M are preferably chosen from titanium, zirconium, boron, aluminium, gallium, indium, thallium, carbon, silicon, germanium, tin and lead, more preferentially from titanium, zirconium, aluminium, carbon, silicon and tin, better still from silicon, aluminium and titanium. Advantageously, the elements M are chosen from silicon and aluminium, better still the element M is silicon.

More preferentially still, the element M precursor is chosen from hexadimethyldisiloxane, tetraethoxysilane, 1,2-bis(triethoxysilyl)ethane, 1,2-bis(trimethoxysilyl)ethane, methoxytrimethylsilane, and mixtures thereof.

According to a first specific embodiment of the invention, the element M is preferably an element from column 4, and better still the element M is titanium.

According to another specific embodiment of the invention, the element M is preferably an element from column 13, and better still the element M is aluminium.

According to another specific embodiment of the invention, the element M is an element from column 14, and better still the element M is silicon.

During the process according to the invention, a (Zinc/M)_{injected} molar atomic ratio can be calculated. This ratio corresponds to the amount in moles of zinc atoms injected during step (b) on the one hand, to the amount in moles of element M injected during step (c) on the other hand.

In particular, when the element M is silicon, the (Zinc/Silicon)_{injected} molar atomic ratio is preferably strictly less than 2.5, more preferentially less than or equal to 2, better still less than or equal to 1.5,and more preferentially still is within the range extending from 0.1 to 1.5, and better still from 0.5 to 1.

When the element M is different from silicon, the (Zinc/M)_{injected} molar atomic ratio is preferably within the range extending from 0.1 to 10, more preferentially from 0.2 to 5.

Preferably, nitrogen is bubbled into composition (B) of the invention, prior to its injection during step (c). The rate of injection of composition (B) can then be controlled by a determination of the pressure known by a person skilled in the art, for instance the method defined by Scott, D. W.; Messerly, J. F.; Todd, S. S.; Guthrie, G. B.; Hossenlopp, I. A.; Moore, R. T.; Osborn, A. G.; Berg, W. T.; McCullough, J. P., Hexamethyldisiloxane: chemical thermodynamic properties and internal rotation about the siloxane linkage, J. Phys. Chem., 1961, 65, 1320-6.

According to one specific embodiment of the invention, composition (B) as described above is, prior to its injection during step (c), brought to a temperature within the range extending from 25° C. to 70° C., more preferentially from 30° C. to 60° C.

Preferably, the content of element M precursor in composition (B) injected during step (c) of the process according to the invention is between 1% and 60% by weight, more preferentially between 5% and 30% by weight, relative to the total weight of the composition (B).

Composition (B) comprises one or more solvents.

Preferably, the solvent(s) present in composition (B) are chosen from polar protic solvents other than water; and more preferentially from (C₁-C₈)alkanols. More preferentially still, composition (B) comprises ethanol.

Preferably, the solvent(s) present in composition (B) are chosen from solvents that are combustible at the flame temperature of step (c), preferably combustible at a temperature between 200° C. and 600° C.; and more preferentially between 300° C. and 400° C. Better still, the solvent(s) present in composition (B) have a boiling point above or equal to room temperature (25° C.), and more preferentially between 50° C. and 120° C.

Preferably, the content of solvent(s) present in composition (B) injected during step (c) of the process according to the invention is between 40% and 99% by weight, more preferentially between 50% and 98% by weight, and better still between 70% and 95% by weight, relative to the total weight of the composition (B).

The preparation process according to the invention may optionally further comprise:
a treatment step (d₁) comprising the introduction of the zinc oxide particles obtained after step (c) into an alkaline bath having a pH of 7 to 11, and preferentially of 7.5 to 9, and/or
a step (d₂) of calcining the zinc oxide particles obtained after step (c) or at the end of treatment step (d₁).

When the treatment step (d₁) is present:

(i) the treatment lasts preferably between 10 and 600 minutes, more preferentially between 40 and 300 minutes; and/or

(ii) the pH of the alkaline bath varies preferably between 7 and 11, more preferentially between 7.5 and 9; and/or

(iii) the temperature is preferably room temperature, i.e. 25° C.; and/or

(iii) the content of particles obtained after step (c) in the alkaline bath is preferably between from 0.5 to 100 g of particles per litre of alkaline bath, more preferentially between 1 and 10 g of particles per litre of alkaline bath.

When the calcining step (d₂) is present:

(i) the calcining lasts preferably between 60 and 400 minutes, more preferentially between 60 and 180 minutes; and/or

(ii) the temperature ranges preferably from 100° C. to 600° C., more preferentially from 100° C. to 300° C., more preferentially still from 130° C. to 250° C.

According to one specific embodiment of the invention, the particles obtained by the preparation process according to the invention are doped. According to this embodiment, composition (A) further comprises one or more precursors of element D, different from element M, with D chosen from fluorine, yttrium, vanadium, scandium, zirconium, hafnium, iron, copper and tungsten.

The Zinc Oxide Particles

Another subject of the invention is a zinc oxide particle, in particular of Zn-M oxide type, comprising a core 1 and one or more upper coating layers 2 covering said core 1, characterized in that:

(i) the core 1 consists of zinc oxide, preferentially in the crystalline state;

(ii) the upper coating layer(s) 2 cover at least 90% of the surface of the core 1, preferably cover the whole of the surface of the core 1, and comprise one or more (in)organic, preferably inorganic, compounds containing one or more elements M and one or more oxygen atoms;

(iii) said element(s) M are chosen from elements from column 4, elements from column 13 and elements from column 14 of the Periodic Table of the Elements; and

it being understood that:
when said element(s) M are silicon then the (Zinc/Silicon)_{particle} molar atomic ratio is strictly less than 2, preferably is within the range extending from 0.1 to 1.5, more preferentially from 0.5 to 1; and
when said element(s) M are different from silicon then the (Zinc/M)_{particle} molar atomic ratio is within the range extending from 0.1 to 10, preferably is within the range extending from 0.1 to 5; and
the BET specific surface area of the particle is between 1 m²/g and 350 m²/g.

The particle according to the invention comprises a core 1 consisting of zinc oxide, preferably in the crystalline state. The crystalline state of the core 1 and also its composition may be, for example, determined by a conventional X-ray diffraction method.

Advantageously, the core 1 of the particle according to the invention consists of one or more aggregates of crystalline primary zinc oxide particles. In other words, the core 1 consists of several microcrystals of zinc oxide.

Preferably, the particle of zinc oxide is obtained by the preparation process of the invention as defined above.

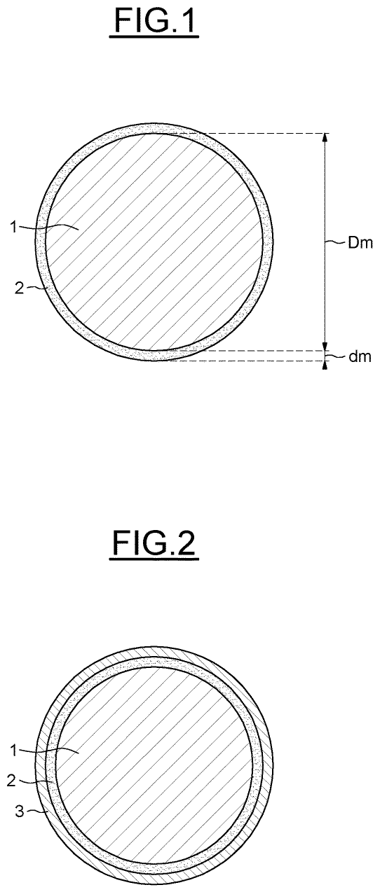

The zinc oxide particle according to FIG. 1 comprises a core 1 of diameter Dₘ, consisting of zinc oxide in the crystalline state and comprising one or more aggregates of primary zinc oxide particles.

The zinc oxide particle according to FIG. 1 also comprises an upper coating layer 2 completely covering the surface of the core 1 and having a thickness dₘ.

The zinc oxide particle according to FIG. 2 corresponds to a zinc oxide particle according to FIG. 1 further comprising an additional coating layer 3 different from the coating layer 2. Said additional coating layer 3 covers at least 90% of the surface of the upper layer 2 and preferably it completely covers the upper coating layer 2.

The number-average diameter Dₘ of the core 1 may, for example, be determined by transmission electron microscopy (abbreviated to TEM). Preferably, the number-average diameter Dₘ of the core 1 of the particle according to the invention is within the range extending from 3 to 1000 nm; more preferentially from 6 to 50 nm, and more preferentially still between 10 and 30 nm.

The zinc oxide particle according to the invention comprises one or more upper coating layers covering at least 90% of the surface of the core.

The degree of coverage of the core by the upper coating layer(s) may for example be determined by means of a visual analysis of TEM-BF or STEM-HAADF type, coupled to a STEM-EDX analysis.

Each of the analyses is carried out on a statistical number of particles, in particular on at least 20 particles. The particles are deposited on a metal grid made of a metal different from zinc, and from any other metal that forms part of the particles, whether in the core or in the upper coating layer(s). For example, the grid is made of copper (except in the case where it is desired to use copper in the manufacture of the particles).

Visual analysis of the TEM-BF and STEM-HAADF images makes it possible, based on the contrast, to deduce whether or not the coating completely surrounds the core of the particle. It is possible, by analysing each of the 20 (or more) images, to deduce a degree of coverage of the core, then, by taking the average, to determine an average degree of coverage.

The STEM-EDX analysis makes it possible to verify that the coating does indeed contain predominantly or exclusively the metal M. For this, it is necessary to make measurements (on at least 20 particles), on the edges of the particles. These measurements then reveal the metal M.

The STEM-EDX analysis also makes it possible to verify that the core does indeed contain the metal zinc. For this, it is necessary to make measurements (on at least 20 particles), at the centres of the particles. These measurements then reveal the metal zinc and the metal M.

Preferably, the upper coating layer(s) 2 completely cover the surface of the core 1.

The number-average thickness dₘ of the upper coating layer(s) 2 may also be determined by transmission electron microscopy. Preferably, the number-average thickness dₘ is within the range extending from 1 to 30 nm; more preferentially from 1 to 15 nm, and more preferentially still from 1 to 6 nm.

Advantageously, the upper coating layer(s) 2 are amorphous.

Preferably, the upper coating layer(s) 2 consist of one or more oxides of the element M. As described above, the element M is chosen from elements from column 4 (titanium column), elements from column 13 (boron column) and elements from column 14 (carbon column) of the Periodic Table of the Elements.

In other words, the elements M are preferably chosen from titanium, zirconium, boron, aluminium, gallium, indium, thallium, carbon, silicon, germanium, tin and lead, more preferentially from titanium, zirconium, aluminium, carbon, silicon and tin, better still from silicon, aluminium and titanium. Advantageously, the elements M are chosen from silicon and aluminium, better still the element M is silicon.

According to a first specific embodiment of the invention, the element M is preferably an element from column 4, and better still the element M is titanium.

According to another specific embodiment of the invention, the element M is preferably an element from column 13, and better still the element M is aluminium.

According to another specific embodiment of the invention, the element M is an element from column 14, and better still the element M is silicon.

Very particularly preferably, the upper coating layer(s) 2 consist of silicon oxide SiO₂, aluminium oxide Al₂O₃, and/or titanium oxide TiO₂; and more preferentially silicon oxide SiO₂.

The zinc oxide particle according to the invention comprises zinc and the element M in a (Zinc/M)_{particle} molar atomic ratio for the particle according to the invention.

This ratio corresponds to the amount in moles of zinc atoms present in the particle according to the invention on the one hand, to the amount in moles of element M present in the particle according to the invention on the other hand.

This ratio can be determined by spectrometry according to one of the following two methods. According to a first method, powder is spread out and an X-ray fluorimetry study is carried out with an X-ray spectrometer to deduce therefrom the metal ratio. According to another method, the particles of the invention are dissolved beforehand in an acid. Then an elemental analysis is carried out on the material obtained by ICP-MS (inductively coupled plasma mass spectrometry) to deduce therefrom the metal ratio.

In particular, when the element M is silicon, the (Zinc/Silicon)_{particle} molar atomic ratio is preferably less than or equal to 1.5, more preferentially said ratio is within the range extending from 0.1 to 1.5, and more preferentially still from 0.5 to 1.

When the element M is different from silicon, the (Zinc/M)_{particle} molar atomic ratio is preferably within the range extending from 0.1 to 5.

Preferably, the sum of the content of zinc oxide and the content of element M oxide is at least equal to 99% by weight, relative to the total weight of the core 1 and of the upper coating layer(s) 2.

The number-average diameter of the particle according to the invention may also be determined by transmission electron microscopy. Preferably, the weight-average diameter of the particle according to the invention is within the range extending from 3 to 1000 nm; more preferentially from 10 to 100 nm, and preferably from 15 to 70 nm.

The BET specific surface area of the particle according to the invention is between 1 m²/g and 350 m²/g; more preferentially between 1 m²/g and 200 m²/g; and even more preferentially between 30 and 100 m²/g.

The zinc oxide particle may optionally further comprise an additional coating layer 3 covering the upper coating layer(s) 2 and preferably comprising one or more hydrophobic organic compounds.

The hydrophobic organic compound(s) are more preferentially chosen from silicones, in particular silicones comprising at least one fatty chain; carbon-based derivatives comprising at least 6 carbon atoms, in particular fatty acid esters; and mixtures thereof.

The additional coating layer 3 may be produced via a liquid method or via a solid method.

Via a liquid method, the hydroxyl functions of the surface of the particles are reacted with reactive functions of the compound which will form the coating (typically silanol functions of a silicone or the acid functions of a carbon-based fatty substance).

Via a solid method, the particles are brought into contact with a liquid or pasty compound comprising the hydrophobic substance. Then, after contact, the mixture is dried and the mixture is crushed, for example by milling.

Another subject of the invention relates to a composition, preferably a cosmetic composition, comprising one or more zinc oxide particles as described above, and preferably obtained by the process according to the invention.

The composition according to the invention is advantageously an aqueous composition.

The coated zinc oxide particle(s) of the invention may also be in dry form (powder, flakes, plates), as a dispersion or as a liquid suspension or as an aerosol. The coated zinc oxide particle(s) of the invention may be used as is or mixed with other ingredients.

The composition of the invention may be in various galenical forms. Thus, the composition of the invention may be in the form of a powder (pulverulent) composition or of a liquid composition, in the form of a milk, a cream, a paste or an aerosol composition.

The composition according to the invention is in particular a cosmetic composition, i.e. the multilayer material(s) of the invention are in a cosmetic medium. The term “cosmetic medium” means a medium that is suitable for application to keratin materials, notably human keratin materials such as the skin, said cosmetic medium generally consisting of water or of a mixture of water and of one or more organic solvents or of a mixture of organic solvents. Preferably, the composition comprises water, in a content notably of between 5% and 95% by weight relative to the total weight of the composition.

The term “organic solvent” means an organic substance that is capable of dissolving another substance without chemically modifying it. As examples of organic solvents that can be used in the composition of the invention, mention may for example by made of lower C₂-C₆ alkanols, such as ethanol and isopropanol; polyols and polyol ethers, for instance 2-butoxyethanol, propylene glycol, propylene glycol monomethyl ether and diethylene glycol monoethyl ether and monomethyl ether, and also aromatic alcohols, for instance benzyl alcohol or phenoxyethanol, and mixtures thereof.

When they are present, the organic solvent(s) are present in proportions preferably between 0.1% and 40% by weight, more preferentially between 1% and 30% by weight and even more particularly between 5% and 25% by weight relative to the total weight of the composition.

The compositions of the invention may contain a fatty phase and may be in the form of direct or inverse emulsions.

The content of the zinc oxide particle(s), present in the composition of the invention, ranges preferably from 0.1% to 40% by weight, more preferentially from 0.5% to 20% by weight, better still from 1% to 10% by weight and more preferentially still from 1.5% to 5% by weight, relative to the total weight of the composition.

According to one specific embodiment of the invention, the composition according to the invention may also be in the form of an anhydrous composition, for instance in the form of an oil. The term “anhydrous composition” is intended to mean a composition containing less than 2% by weight of water, preferably less than 1% by weight of water, and even more preferentially less than 0.5% by weight of water relative to the total weight of the composition, or even a composition that is free of water. In compositions of this type, the water possibly present is not added during the preparation of the composition, but corresponds to the residual water provided by the mixed ingredients.

The composition according to the invention may be prepared according to the techniques that are well known to those skilled in the art. It may in particular be in the form of a simple or complex emulsion (oil-in-water, or abbreviated to O/W, water-in-oil or W/O, oil-in-water-in-oil or O/W/O, or water-in-oil-in-water or W/O/W), such as a cream, a milk or a cream gel, or else in powder form or in the form of an aerosol composition.

Another subject of the invention is the composition according to the invention, preferably a cosmetic composition, for use for protecting the skin, preferably human skin, against visible radiation (i.e. wavelengths between 400 nm and 800 nm) and/or ultraviolet radiation (i.e. wavelengths between 100 nm and 400 nm), UV-A radiation (i.e. wavelengths between 320 nm and 400 nm) and/or UV-B radiation (i.e.

wavelengths between 280 nm and 320 nm). The compositions according to the invention make it possible to screen out solar radiation efficiently, with a broad spectrum, in particular for UV-A radiation (including long-wave UV-A radiation), while being particularly stable over time under UV exposure.

The composition according to the present invention may optionally comprise one or more additional UV-screening agents, other than the zinc oxide particle according to the invention, chosen from hydrophilic, lipophilic or insoluble organic UV-screening agents and/or one or more mineral pigments. It will preferentially be constituted of at least one hydrophilic, lipophilic or insoluble organic UV-screening agent.

Another subject of the invention is the use of the zinc oxide particles as described above, and preferably obtained by the process according to the invention:
for formulating cosmetic or pharmaceutical compositions, in particular intended to protect the skin, in particular human skin, against visible and/or ultraviolet radiation or to modify the appearance of the skin, in particular human skin,
for formulating paints, varnishes and/or stains, or
for manufacturing a coating for electronic devices or products, notably for obtaining moisture-resistant electronic components.

The coated zinc oxide particle(s) of the invention are preferably an agent for protecting against UVA and UVB radiation. They may notably improve the overall screening-out of UV radiation while maintaining a good overall transmission in the visible range and an excellent transparency in the visible range (400-780 nm).

Application Process

Another subject of the invention is a process for treating keratin materials, notably human keratin materials such as the skin, by application to said materials of a composition as defined previously, preferably by 1 to 5 successive applications, leaving to dry between the layers, the application(s) being sprayed or otherwise.

The compositions of the invention may be used in single application or in multiple application. When the compositions of the invention are intended for multiple application, the content of particles of element M oxide of the invention is generally lower than in compositions intended for single application.

For the purposes of the present invention, the term “single application” means a single application of the composition, this application possibly being repeated several times per day, each application being separated from the next one by one or more hours, or an application once a day, depending on the need.

For the purposes of the present invention, the term “multiple application” means application of the composition repeated several times, in general from 2 to 5 times, each application being separated from the next one by a few seconds to a few minutes. Each multiple application may be repeated several times per day, separated from the next one by one or more hours, or each day, depending on the need.

They may also be connected application methods, such as a saturated single application, i.e. the single application of a cosmetic composition with a high concentration of zinc oxide particles coated with silicon oxide according to the invention, or else with multiple applications of cosmetic composition (less concentrated) comprising one or more zinc oxide particles coated with silicon oxide according to the invention. In the case of multiple applications, several successive applications of cosmetic compositions comprising one or more zinc oxide particles coated with silicon oxide of the invention may be repeated with or without a delay between the applications.

According to one embodiment of the invention, the multiple application is performed on the keratin materials with a drying step between the successive applications of the cosmetic compositions comprising the zinc oxide particle(s) coated with silicon oxide according to the invention. The drying step between the successive applications of the cosmetic compositions comprising one or more zinc oxide particles coated with silicon oxide according to the invention may be performed in the open air or artificially, for example with a hot air drying system such as a hairdryer.

Another subject of the invention is the use of one or more zinc oxide particles coated with silicon oxide according to the invention as defined above as UVA and UVB screening agent to protect keratin materials, notably human keratin materials, such as the skin.

The examples that follow serve to illustrate the invention without, however, being limiting in nature.

### EXAMPLES

### Example 1

1.1 Firstly, a composition (A) of zinc naphthenate (500 mM) in xylene was prepared.

Uncoated zinc oxide particles P1 were then prepared using a conventional FSP preparation process Prep 1 with the pre-prepared composition (A) (outside the invention).

Next, zinc oxide particles coated with silicon dioxide P2 were then prepared using the preparation process Prep 2 according to the invention with the same composition (A) and a composition (B) comprising hexadimethyldisiloxane and ethanol in a proportion of 3:1 (invention).

The parameters of the Prep 1 process are the following:
ratio (composition (A)/O₂)=5 mL/min of liquid and 7 L/min of gas (O₂). To adjust the oxygen flow rate, φ=0.48 is used.

The parameters of the Prep 2 process are the following:
ratio (composition (A)/O₂)=5/7, i.e. 5 mL/min of liquid and 7 L/min of gas (O₂). To adjust the oxygen flow rate, φ=0.48 is used.

In this Prep 2 process, a 40 cm high quartz tube is used. Furthermore, nitrogen is first bubbled through the composition (B). When the composition (B) is injected, the stream of nitrogen heated to 25° C. is adjusted in order to enable the evaporation of the hexadimethyldisiloxane (HMDSO) and so that the (Zn/Si)_{injected} ratio=1.

1.2 Once the particles had been prepared, it was observed that the zinc oxide particles obtained were crystalline.

Furthermore, the particles obtained according to process Prep 2 according to the invention are coated with silicon dioxide and have a (Zn/Si)_{particle} atomic ratio of 1.

The BET specific surface area of the particles according to process Prep 2 is 74 m²/g.

The particles according to process Prep 2 have a number-average diameter equal to 40 nm.

1.3 Evaluation of the Water Resistance:

A first aqueous suspension S1 was prepared from particles P1 and water in a content of 200 mg of P1/L of water.

In the same way, a second aqueous suspension S2 was prepared from particles P2 and water in a content of 200 mg of P2/L of water.

Next, each of the suspensions S1 and S2 were placed in an ultrasound bath for 10 min at a power of 20 W.

Then, a fraction of the suspension S1 and a fraction of the suspension S2 were brought to pH=5 by means of a nitric acid solution.

The content of Zn²⁺ present in the suspensions as a function of time, and relative to the amount of zinc introduced, is then measured by means of a conventional anodic stripping voltammetry method for each suspension.

The results have been collated in the table below:

| | Content of Zn²⁺ (% ions released in a litre of water) | | | | |
|---|---|---|---|---|---|
| Suspensions | at t₀ | at t₀ + 1 h | at t₀ + 2 h | at t₀ + 3 h | at t₀ + 4 h |
| S1 at pH 8 | 0 | 92 | 95 | 98 | 98 |
| (comparative) | | | | | |
| S1 at pH 5 | 0 | 95 | >99 | >99.5 | >99.5 |
| (comparative) | | | | | |
| S2 at pH 8 | 0 | 0.5 | 0.8 | 1.2 | 1.5 |
| (invention) | | | | | |
| S2 at pH 5 | 0 | 1.2 | 2.2 | 4.6 | 6.2 |
| (invention) | | | | | |

t₀ corresponds to the first measurement carried out less than 10 min after the end of the ultrasound bath.

It should be noted that the coated zinc oxide particles P2 obtained according to the preparation process Prep 2 according to the invention have a much better water resistance than the uncoated zinc oxide particles P1 obtained according to the comparative preparation process Prep 1. The uncoated particles P1 are almost eliminated in less than 1 hour and cannot therefore be used in a composition comprising water or if the coating is brought into contact with water.

### Example 2

An aqueous suspension S3 was prepared from zinc oxide particles sold under the reference Z-COTE HP1 (Oxide and Triethoxycaprylylsilane) by the company BASF and water in a content of 200 mg of commercial ZnO/L of water. In other words, the particles Z-COTE HP1 are coated with a layer of triethoxycaprylylsilane, thus giving them a hydrophobic property and protection from water. The BET specific surface area of these particles is 15.8 m²/g.

Next, the suspension S3 was placed in an ultrasound bath for 10 min at a power of 20 W.

The content of Zn²⁺ present in the suspension S2 (at pH=8) as prepared in example 1 above and in the suspension S3 (at pH=8), as a function of time, and relative to the amount of zinc introduced, is then measured by means of a conventional anodic stripping voltammetry method for each suspension.

The results have been collated in the table below:

| | Content of Zn²⁺ (concentration of | | | |
|---|---|---|---|---|
| | ions released in a litre of water (ppb)) | | | |
| Suspensions | at t₀ | at t₀ + 2 h | at t₀ + 24 h | at t₀ + 48 h |
| S2 at pH 8 | 0 | 90 | 105 | 135 |
| (invention) | | | | |
| S3 at pH 8 | 0 | 790 | 1550 | 2450 |
| (comparative) | | | | |

t₀ corresponds to the first measurement carried out less than 10 min after the end of the ultrasound bath.

It should be noted that the coated zinc oxide particles P2 obtained according to the preparation process Prep 2 according to the invention have a much better water resistance than the commercial zinc oxide particles, despite an especially high BET specific surface area (74 m²/g for the particles derived from Prep 2 versus 15.8 m²/g for the commercial compound).

2.4. Evaluation of the Optical Properties

Aqueous solutions S′1 and S′2 were prepared from the particles P1 and P2 prepared in Example 1 and an aqueous solution S′3 was prepared from the zinc oxide particles sold under the reference Z-COTE HP1. In each of the solutions, the content of particles represents 0.3% by weight, whilst the remainder is a water/propylene glycol (50/50) mixture.

The transmittance is obtained by exposing the sample to a luminous flux then performing the ratio between the transmitted intensity and the incident intensity.

T=I/I₀

The results have been collated in the table below:

| | Transmittance | | | |
|---|---|---|---|---|
| Suspensions | at 350 nm | at 315 nm | at 430 nm | at 510 nm |
| S′1 (comparative) | 0.21 | 0.20 | 0.86 | 0.92 |
| S′2 (invention) | 0.20 | 0.195 | 0.86 | 0.92 |
| S′3 (comparative) | 0.20 | 0.19 | 0.60 | 0.68 |

The above results show that the transmittances of uncoated zinc particles (P1), particles according to the invention (P2) and commercial particles are similar in the UVA radiation zone, despite a zinc concentration two times lower for the particles of the invention (ZnO/SiO₂, P2) than for the comparative uncoated zinc oxide particles (ZnO, P1).

Furthermore, the zinc oxide particles according to the invention (P2) and the uncoated particles (P1) have a high-level transmittance in the visible spectrum, much better than that of the commercial product.

Thus, the zinc oxide particles of the invention make it possible to obtain a better water resistance, a better transparency in the visible spectrum, while retaining good optical properties.

### Example 3

Zinc oxide particles coated with alumina P3 were prepared using the preparation process Prep 2 according to the invention with composition (A) and a composition (C) comprising the aluminium precursor (aluminium tri-sec-butoxide, C₁₂H₂₇AlO₃) and xylene in a proportion of 3:1.

The parameters of the Prep 2 process are the following:
ratio (composition (A)/O₂)=5/7, i.e. 5 mL/min of liquid and 7 L/min of gas (O₂). To adjust the oxygen flow rate, φ=0.48 is used.

In this Prep 2 process, a 40 cm high quartz tube is used. Furthermore, nitrogen is first bubbled through the composition (C). When the composition (C) is injected, the stream of nitrogen heated to 40° C. is adjusted in order to enable the evaporation of the aluminium tri-sec-butoxide, so that the (Zn/Al)_{injected} ratio=1.

Once the particles had been prepared, it was observed that the zinc oxide particles obtained were crystalline.

Furthermore, the particles obtained according to process Prep 2 according to the invention are coated with Al₂O₃ and have a (Zn/Al)_{particle} atomic ratio of 1.

The BET value of the particles according to process Prep 2 is 60 m²/g.

The particles have a size: 36 nm

### Example 4

Zinc oxide particles coated with tin dioxide P4 were prepared using the preparation process Prep 2 according to the invention with the same composition (A) and a composition (D) comprising the tin precursor (tin ethylhexanoate, C₁₆H₃₀O₄Sn) in xylene in a proportion of 3:1.

The parameters of the Prep 2 process are the following:
ratio (composition (A)/O₂)=5/7, i.e. 5 mL/min of liquid and 7 L/min of gas (O₂). To adjust the oxygen flow rate, φ=0.48 is used.

In this Prep 2 process, a 40 cm high quartz tube is used. Furthermore, nitrogen is first bubbled through the composition (D). When the composition (D) is injected, the stream of nitrogen heated to 50° C. is adjusted in order to enable the evaporation of the tin ethylhexanoate, so that the (Zn/Sn)_{injected} ratio=1.

Once the particles had been prepared, it was observed that the zinc oxide particles obtained were crystalline.

Furthermore, the particles obtained according to process Prep 2 according to the invention are coated with tin dioxide and have a (Zn/Sn)_{particle} atomic ratio of 1.

The BET value of the particles according to process Prep 2 is 72 m²/g.

### Example 5

Zinc oxide particles coated with titanium dioxide P5 were prepared using the preparation process Prep 2 according to the invention with the same composition (A) and a composition (E) comprising the titanium precursor (titanium isopropoxide, C₁₂H₂₈O₄Ti) and xylene in a proportion of 3:1.

The parameters of the Prep 2 process are the following:
ratio (composition (A)/O₂)=5/7, i.e. 5 mL/min of liquid and 7 L/min of gas (O₂). To adjust the oxygen flow rate, φ=0.48 is used.

In this Prep 2 process, a 40 cm high quartz tube is used. Furthermore, nitrogen is first bubbled through the composition (E). When the composition (E) is injected, the stream of nitrogen heated to 50° C. is adjusted in order to enable the evaporation of the titanium isopropoxide, so that the (Zn/Ti)_{injected} ratio=1.

Once the particles had been prepared, it was observed that the zinc oxide particles obtained were crystalline.

Furthermore, the particles obtained according to process Prep 2 according to the invention are coated with titanium dioxide and have a (Zn/Ti)_{particle} atomic ratio of 1.

The BET value of the particles according to process Prep 2 is 40 m²/g.

A RAMAN study of the particles P1 to P5 was carried out. The Raman peak of the ZnO of the particles P1 to P5 was observed.

1. Process for preparing coated zinc oxide particles, in particular of Zn-M oxide type, characterized in that it comprises at least the following steps:
a. preparing a composition (A) by adding one or more zinc precursors to a combustible solvent or to a mixture of combustible solvents; then
b. in a flame spray pyrolysis device, forming a flame by injecting the composition (A) and an oxygen-containing gas until aggregates of zinc oxide are obtained; and
c. injecting into the flame a composition (B) comprising one or more precursors of element M and one or more solvents until an (in)organic, preferably inorganic, coating layer containing at least one element M and at least one oxygen atom is obtained on the surface of said zinc oxide aggregates; said element M being chosen from elements from column 4, elements from column 13 and elements from column 14 of the Periodic Table of the Elements.

2. Process according to claim 1, characterized in that the zinc precursor comprises one or more zinc atoms optionally complexed to one or more ligands containing at least one carbon atom; preferably said ligand(s) are chosen from the following groups: acetate, (C₁-C₆)alkoxylate, (di)(C₁-C₆)alkylamino, and arylate such as naphthalate or naphthenate.

3. Process according to claim 1, characterized in that the combustible solvent(s) are chosen from protic combustible solvents, aprotic combustible solvents, and mixtures thereof; preferably from alcohols, esters, acids, acyclic ethers, cyclic ethers, aromatic hydrocarbons or arenes, non-aromatic hydrocarbons, and mixtures thereof; more preferentially, the combustible solvent(s) are chosen from aprotic combustible solvents comprising at least three carbon atoms and mixtures thereof; better still from xylene, tetrahydrofuran, 2-ethylhexyl acetate, 2-ethylhexanoic acid (EHA), and mixtures thereof.

4. Process according to claim 1, characterized in that the content of zinc precursor in the composition (A) is between 1% and 60% by weight, preferably between 15% and 30% by weight, relative to the total weight of the composition (A).

5. Process according to claim 1, characterized in that the flame formed in step (b) and maintained in step (c) is, at the outlet of the tube transporting the composition (B), at a temperature between 200° C. and 600° C.; preferably between 300° C. and 400° C.

6. Process according to claim 1, characterized in that the element(s) M are chosen from titanium, zirconium, boron, aluminium, gallium, indium, thallium, carbon, silicon, germanium, tin and lead; preferably from titanium, zirconium, aluminium, carbon, silicon and tin; more preferentially from silicon, aluminium and titanium; better still from silicon and aluminium; more preferentially still the element M is silicon.

7. Process according to claim 1, characterized in that the precursor of element M comprises at least two M atoms and several M—carbon covalent bonds; preferably, the precursor of element M comprises at least three M atoms and several M—carbon covalent bonds; more preferentially, the precursor of element M is chosen from hexadimethyldisiloxane, tetraethoxysilane, 1,2-bis(triethoxysilyl)ethane, 1,2-bis(trimethoxysilyl)ethane, methoxytrimethylsilane, and mixtures thereof.

8. Process according to claim 1, characterized in that the element M is silicon and the (Zinc/Silicon)_{injected} molar atomic ratio is strictly less than 2.5, preferably less than or equal to 2, more preferentially less than or equal to 1.5, better still is within the range extending from 0.1 to 1.5, and more preferentially still from 0.5 to 1.

9. Process according to claim 1, characterized in that the element M is different from silicon and the (Zinc/M)_{injected} molar atomic ratio is within the range extending from 0.1 to 10, preferably from 0.2 to 5.

10. Process according to claim 1, characterized in that the composition (B) comprises one or more solvents chosen from polar protic solvents other than water; more preferentially from (C₁-C₈)alkanols; and better still the solvent is ethanol.

11. Process according to claim 1, characterized in that the content of precursor of element M in the composition (B) is between 1% and 60% by weight, preferably between 5% and 30% by weight, relative to the total weight of the composition (B).

12. Process according to claim 1, characterized in that it further comprises a treatment step (di) comprising the introduction of the zinc oxide particles obtained after step (c) into an alkaline bath having a pH of 7 to 11, and/or a step of calcining (d₂) the zinc oxide particles obtained after step (c) or at the end of the treatment step (d₁).

13. Zinc oxide particle, in particular of Zn-M oxide type, comprising a core (1) and one or more upper coating layers (2) covering said core (1), characterized in that:
the core (1) consists of zinc oxide, preferably in the crystalline state;
(ii) said upper coating layer(s) (2) cover at least 90% of the surface of the core (1), preferably cover the whole of the surface of the core (1), and comprise one or more (in)organic, preferably inorganic, compounds, containing one or more elements M and one or more oxygen atoms;
(iii) said element(s) M are chosen from elements from column 4, elements from column 13 and elements from column 14 of the Periodic Table of the Elements; and
it being understood that:
when said element(s) M are silicon then the (Zinc/Silicon)_{particle} molar atomic ratio is strictly less than 2, preferably is within the range extending from 0.1 to 1.5, more preferentially from 0.5 to 1;
when said element(s) M are different from silicon then the (Zinc/M)_{particle} molar atomic ratio is within the range extending from 0.1 to 10, preferably is within the range extending from 0.1 to 5; and
the BET specific surface area of said particle is between 1 m²/g and 350 m²/g.

14. Particle obtained by the process as defined in claim 1.

15. Particle according to claim 13, characterized in that the upper coating layer(s) (2) consist of one or more oxides of element M; preferably, the upper coating layer(s) (2) consist of silicon oxide SiO₂, aluminium oxide Al₂O₃, and/or titanium oxide TiO₂; and more preferentially silicon oxide SiO₂.

16. Particle according to claim 15, characterized in that the sum of the content of zinc oxide and the content of element M oxide is at least equal to 99% by weight, relative to the total weight of the core (1) and of the upper coating layer(s) (2).

17. Particle according to claim 13, characterized in that the number-average diameter Dₘ of the core (1), determined by transmission electron microscopy (TEM), is within the range extending from 3 to 1000 nm, preferably from 6 to 50 nm, and more preferentially from 10 to 30 nm.

18. Particle according to claim 13, characterized in that the number-average thickness dₘ of the upper coating layer(s) (2), measured by transmission electron microscopy (TEM), is within the range extending from 1 to 30 nm, preferably from 1 to 15 nm, and more preferentially from 1 to 6 nm.

19. Particle according to claim 13, characterized in that the number-average diameter of the particle, determined by transmission electron microscopy (TEM), is within the range extending from 3 to 1000 nm, preferably from 10 to 100 nm, and more preferentially from 15 to 70 nm.

20. Particle according to claim 13, characterized in that it further comprises an additional coating layer (3) covering the upper coating layer(s) (2); and said additional layer preferably comprising one or more hydrophobic organic compounds, more preferentially chosen from silicones, carbon-based derivatives comprising at least 6 carbon atoms, and mixtures thereof.

21. Composition comprising one or more zinc oxide particles as defined in claim 13.

22. Composition as defined in claim 21 for use for protecting the skin, preferably human skin, against visible and/or UV-A and/or UV-B ultraviolet radiation.

23. Use of the zinc oxide particles as defined in claim 13:
for formulating cosmetic or pharmaceutical compositions, in particular intended to protect the skin against visible and/or ultraviolet radiation or to modify the appearance of the skin,
for formulating paints, varnishes and/or stains, or
for manufacturing a coating for electronic devices or products, notably for obtaining moisture-resistant electronic components
